(19) United States
(12) Patent Application Publication
(10) Pub. No.: US 2025/0018169 A1
(43) Pub. Date: Jan. 16, 2025

(54) CATHETER BLOOD PUMP WITH DEPLOYABLE INTRA-CARDIAC ANTI-MIGRATION BRACE

(71) Applicant: FBR Medical, Inc., El Dorado Hills, CA (US)

(72) Inventors: Peter G. Piferi, Orange, CA (US); Jesus Flores, Mission Viejo, CA (US)

(21) Appl. No.: 18/712,047

(22) PCT Filed: Nov. 30, 2022
(86) PCT No.: PCT/US2022/080608

§ 371 Date: May 21, 2024

Related U.S. Application Data
(60) Provisional application No. 63/287,396, filed on Dec. 8, 2021.

(51) Int. Cl. A61M 60/139; A61M 60/221; A61M 60/411

(52) U.S. Cl. CPC A61M 60/139; A61M 60/221; A61M 60/411

(57) ABSTRACT

Catheter blood pumps that have an impeller, an outlet cage at least partially surrounding the impeller, a drive cable operatively coupled to a motor at a proximal end thereof, and operatively coupled to the impeller at a distal end thereof, a catheter, wherein the drive cable is disposed within the catheter and an anti-migration brace coupled to a component of the catheter blood pump. The anti-migration brace is configured to have a first configuration during a tortuous insertion of the catheter into a patient and a deployable second configuration that is larger than the first configuration when in position in target anatomy of the patient whereby the anti-migration brace expands laterally outwardly in size from the first configuration to the second configuration.

### RELATED APPLICATIONS

This patent application claims the benefit of and priority to U.S. Provisional Patent Application Ser. No. 63/287,396 filed Dec. 8, 2021, the contents of which are hereby incorporated by reference as if recited in full herein.

### FIELD OF THE INVENTION

This invention relates generally to catheter blood pumps.

### BACKGROUND OF THE INVENTION

Some patients who have heart failure, and some of those at risk for developing it, receive interventions intended to temporarily assist the heart before or during a medical or surgical procedure and/or during a recovery period. The intervention typically lasts for less than a week but can continue for several weeks. These interventions include pharmaceuticals and/or medical devices, including cardiac-assist devices.

Some cardiac-assist devices include a pump to supplement the heart's pumping action. By assuming some of the heart's pumping function, these “blood pumps” unload the heart, helping it to recover. Cardiac-assist devices can be temporary or permanent.

Some blood pumps are percutaneous with the impeller (and in some devices, the pump's motor) residing within the patient. These blood pumps are often coupled to a catheter and are consequently referred to as “catheter blood pumps.” Some catheter blood pumps are inserted into the patient using established cath-lab techniques, wherein they are advanced through the vascular system (typically entering through the femoral artery or the radial artery) to a patient's heart. This approach is significantly less invasive than cardiac surgery or other relatively complicated procedures.

Over the years, various types of blood pumps have been developed for the purpose of augmenting or replacing the blood pumping action of damaged or diseased hearts. The pumps may be designed to provide right and/or left ventricular assist, although left ventricle assist is the most common application in that it is far more common for the left ventricle to become diseased or damaged than it is for the right ventricle.

Blood pumps must pump the fluid at a suitable rate without applying excessive Reynolds shear stress to the fluid. It is well known to those skilled in the art that lysis or cell destruction may result from application of shear stress to cell membranes. Red blood cells are particularly susceptible to shear stress damage as their cell membranes do not include a reinforcing cytoskeleton to maintain cell shape.

Intravascular blood pumps capable of being percutaneously or surgically introduced into the vascular system of a patient typically provide left and/or right heart support. See, e.g., U.S. Pat. No. 4,625,712 which describes a multiple stage intravascular axial-flow blood pump which can be percutaneously inserted into an artery for heart assist and U.S. Pat. No. 4,846,152 which describes a single-stage intravascular axial flow blood pump, the contents of which are hereby incorporated by reference as if recited in full herein. These blood pumps position the drive unit/motor outside the body (extracorporeal) and use long cable drive systems. The maneuverability and/or durability of these types of blood pumps was often less than desired. During use, components of these devices tended to deteriorate prematurely due to rotational and pulsatile forces experienced by the blood pumps.

Other intravascular blood pumps are configured so that the drive unit/motor and the impeller are directly connected to each other, with the motor and the impeller (pump) housing having the substantially the same outer diameter. See, e.g., U.S. Pat. No. 6,176,848, the contents of which are hereby incorporated by reference as if recited in full herein. While these systems have been used successfully to pump blood, the flow rates provided are typically under 3-4 liters/minute at a counterpressure of about 100 mm Hg. The pumping rate can be limited by the low torque limitation of the small “micro” motors.

Because a catheter blood pump is not surgically implanted in a patient and it can be desirable to temporarily be fixed in a desired location in the patient's anatomy to avoid migration due to pulsatile cardiac activity. For example, in some embodiments, it is desirable to position the catheter blood pump across the aortic valve. In the absence of some way to stabilize the pump, it may migrate out of position, e.g., out of the ventricle.

### SUMMARY

Some embodiments of the present invention provide a catheter blood pump including a deployable anti-migration brace. The brace is able to prevent the distal end portion of catheter blood pump from migrating into the aorta once positioned in a target anatomical position, until such time as the catheter blood pump is to be removed.

The deployable brace can be in the form of an expandable mesh.

The deployable brace can be in the form of an expandable full or partial loop shape having a first configuration prior to deployment and a second configuration after deployment.

The deployable brace can have a three-dimensional shape after deployment.

The deployable brace can be sized and configured to sufficiently expand from the pre-deployment configuration to the deployed configuration to abut the wall(s) of anatomy in which it resides, such as the ascending aorta.

The deployable brace can be provided by a tube fixedly attached to the catheter with a plurality of circumferentially spaced apart and longitudinally extending slits formed in the tube and defining outwardly deployable fingers.

The deployable brace can have a spiral member that has a radial extent that is greater than an outer diameter of a catheter body.

Embodiments of the present invention are directed to a catheter blood pump that includes: an impeller; an outlet cage at least partially surrounding the impeller; a drive cable operatively coupled to a motor at a proximal end of the drive cable and operatively coupled to the impeller at a distal end of the drive cable and a catheter. The drive cable is disposed within the catheter. The catheter blood pump also includes an anti-migration brace coupled to a component of the catheter blood pump. The anti-migration brace is configured to have a first configuration during a tortuous insertion of the catheter into a patient and a deployable second configuration that is larger than the first configuration when in position in target anatomy of the patient whereby the anti-migration brace expands laterally outwardly in size from the first configuration to the second configuration.

In the second configuration, the anti-migration brace can increase in size in a longitudinal direction from a first position to a second more distal position closer to the impeller.

The anti-migration brace can be disposed on an exterior of the catheter, proximal to the outlet cage.

The catheter blood pump can further include a sheath enclosing the anti-migration brace when the anti-migration brace is in the first configuration. The sheath can be configured to force the anti-migration brace against the component of the catheter blood pump and provide a profile of 6F-18F in the first configuration.

The catheter can have a multi-lumen catheter body and can include at least one longitudinally extending brace lumen. A distal end portion of the anti-migration brace can be configured to be extendable out of the at least one longitudinally extending brace lumen and retractable back into the at least one longitudinally extending brace lumen.

The anti-migration brace can be attached to an outer surface of the catheter at a location that is a distance proximal to the impeller and can be sized and configured to reside about an aortic wall of an ascending aorta of the patient in the second configuration.

The anti-migration brace can have a maximal projection at a location that can be in a range of 5 mm-4 cm proximal to a proximal end of the impeller.

The anti-migration brace can be attached to an impeller housing distal to the outlet cage.

The anti-migration brace can be attached to an impeller housing proximal to the outlet cage.

The anti-migration brace can have an expandable mesh body with proximal and distal end portions configured to attach to the component of the catheter blood pump and a medial segment therebetween configured to expand into a three-dimensional shape in the second configuration.

The anti-migration brace can be an expandable lasso.

The expandable lasso can have a single loop.

The expandable lasso can have an open loop configuration.

The anti-migration brace can have a plurality of fingers that can project radially outward from the catheter when deployed to the second configuration.

The fingers can be circumferentially spaced apart and attached to a tube segment, and the tube segment can be attached to the catheter.

The fingers can have a non-deployed first configuration whereby the fingers define a cylinder aligned with the tube segment.

The anti-migration brace can be provided by a tubular body with first and second circumferentially spaced apart and longitudinally extending slits separate a respective finger.

The plurality of fingers can be provided in a number of 3 to 8.

The anti-migration brace can have a spiral member with a series of circumferential segments that increase in radius from a proximal end to a more distal end and that have radial extents that are greater than an outer diameter of a catheter body.

The anti-migration brace can have an atraumatic configuration with tissue contact surfaces that are devoid of tissue hooks.

On introduction of the catheter blood pump into the patient, the sheath, where used, can contain the at least part of the catheter and the anti-migration brace. The anti-migration brace can be physically adapted to automatically expand when the sheath is partially slidably withdrawn relative to the catheter or the catheter is partially slidably extended relative to the sheath.

Further features, advantages and details of the present invention will be appreciated by those of ordinary skill in the art from a reading of the figures and the detailed description of the preferred embodiments that follow, such description being merely illustrative of the present invention.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim or file any new claim accordingly, including the right to be able to amend any originally filed claim to depend from and/or incorporate any feature of any other claim although not originally claimed in that manner. These and other objects and/or aspects of the present invention are explained in detail in the specification set forth below.

### BRIEF DESCRIPTION OF THE DRAWINGS

FIG. 1A is a partial section view of a catheter blood pump with an anti-migration brace in a first, non-deployed configuration according to embodiments of the present invention.

FIG. 1B is a partial section view of the catheter blood pump shown in FIG. 1A with the anti-migration brace in an example deployed configuration according to embodiments of the present invention.

FIG. 2A is a partial section view of the catheter blood pump shown and anti-migration brace shown in FIG. 1A, shown rotated 90 degrees.

FIG. 2B is a partial section view of the catheter blood pump shown in FIG. 2A with the anti-migration brace in the example deployed configuration according to embodiments of the present invention.

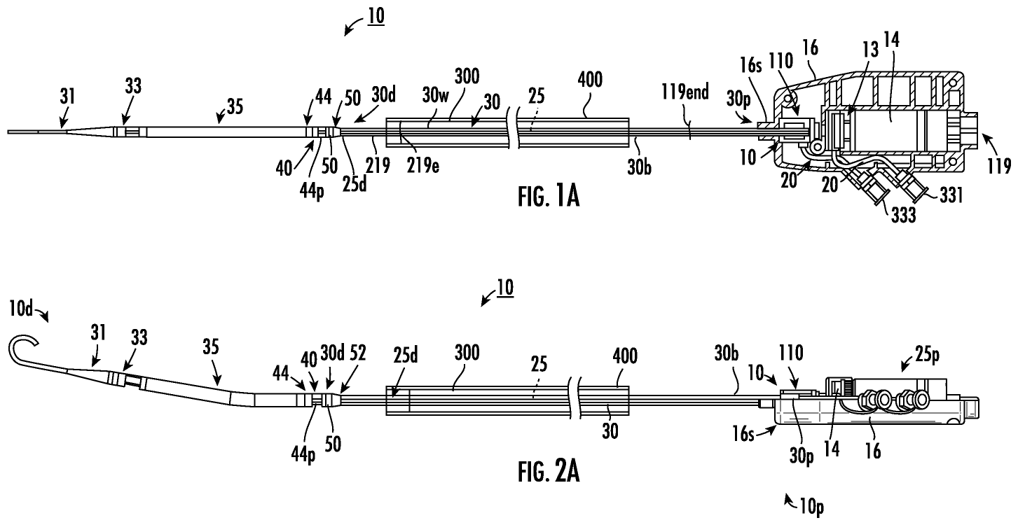

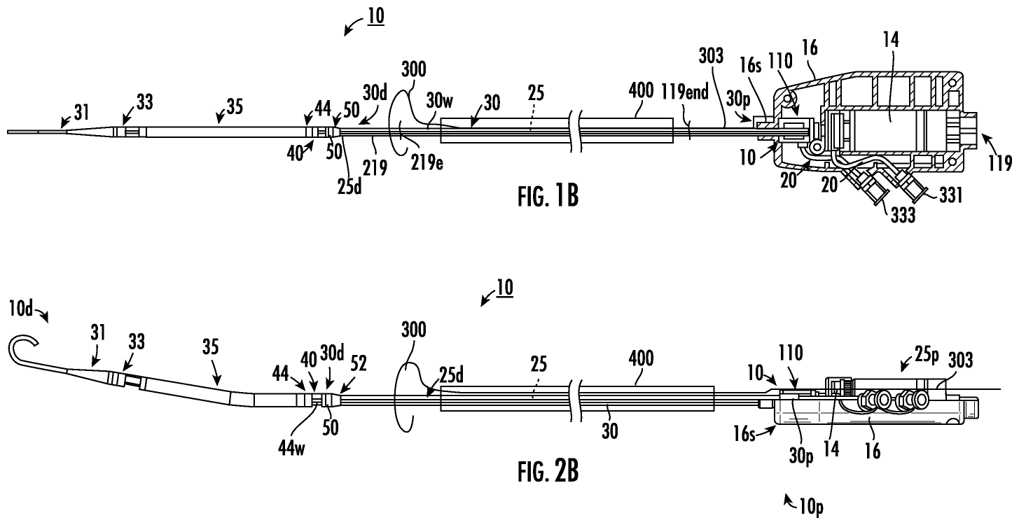

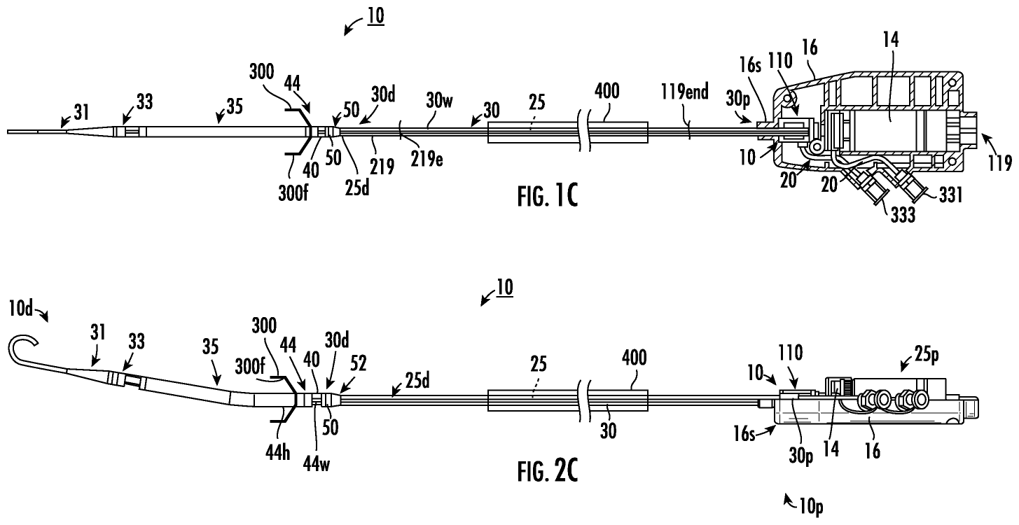

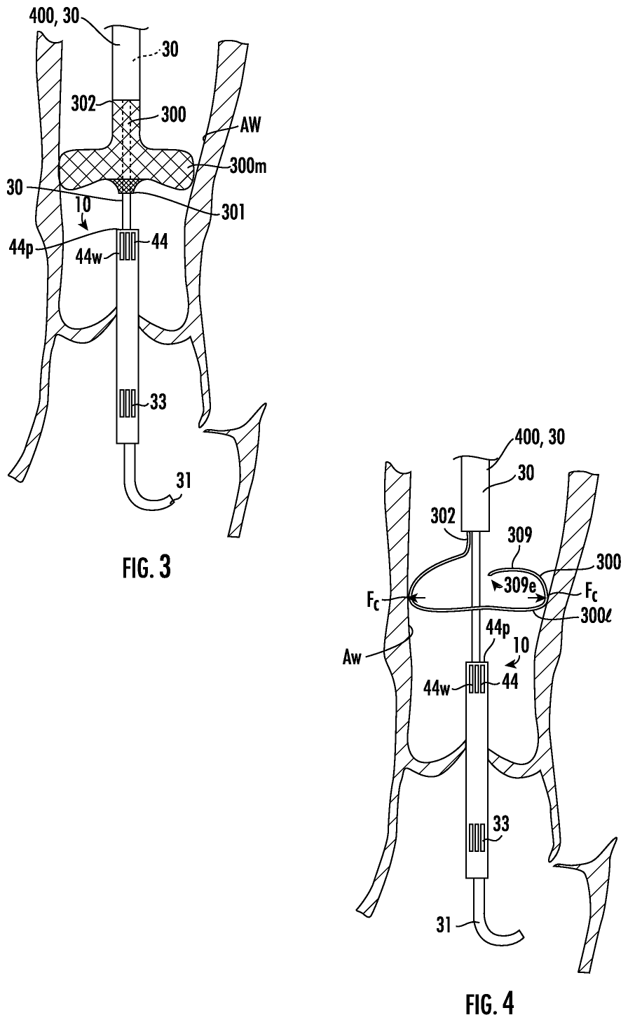

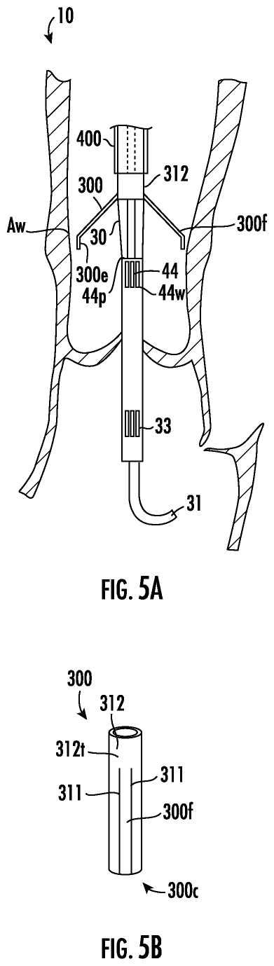

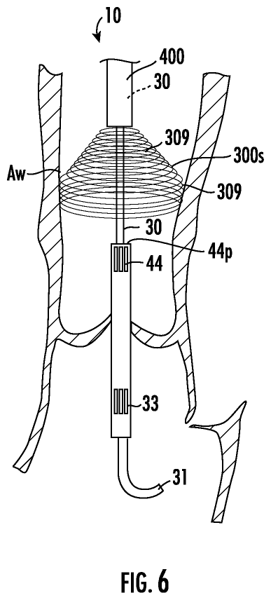

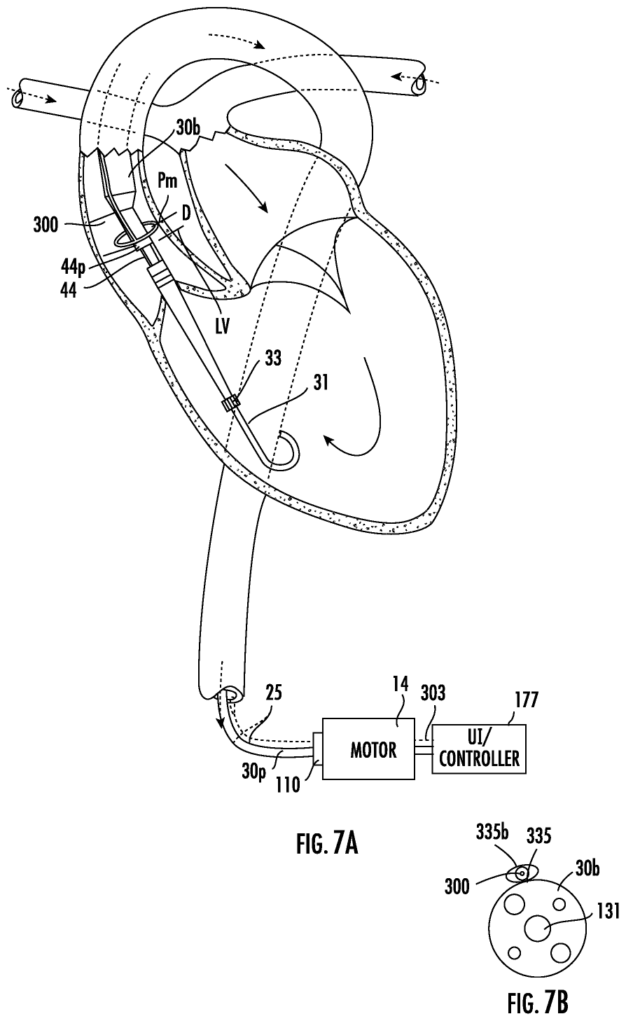

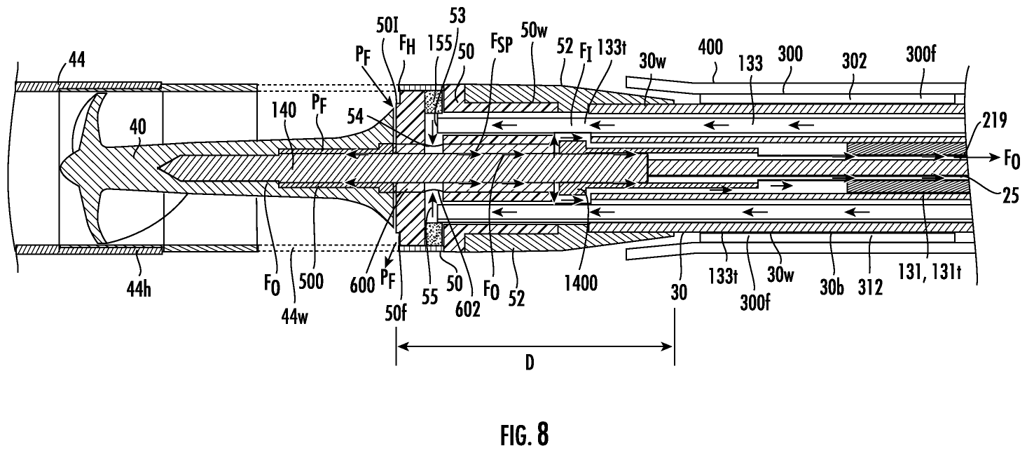

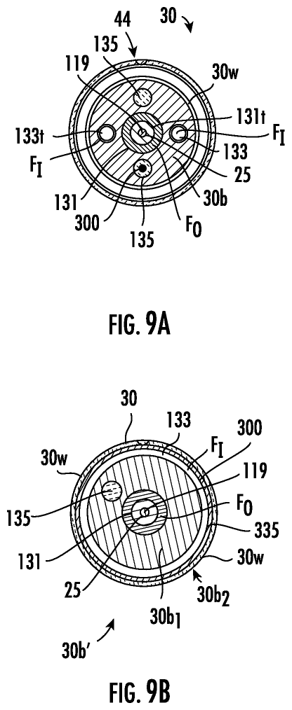

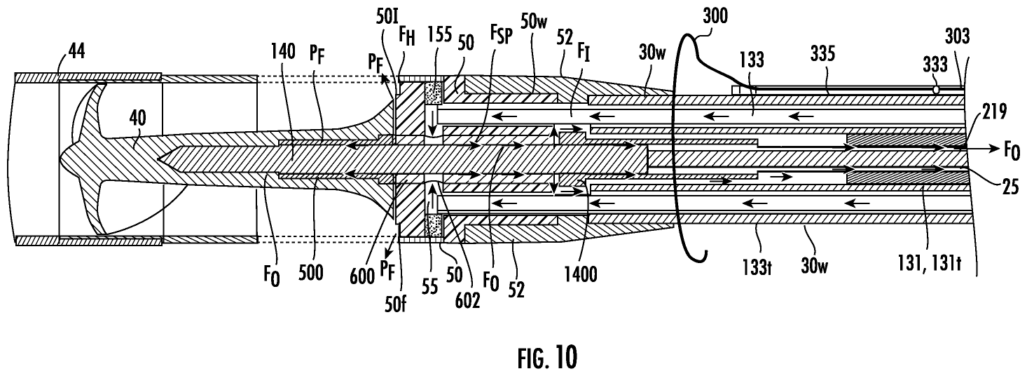

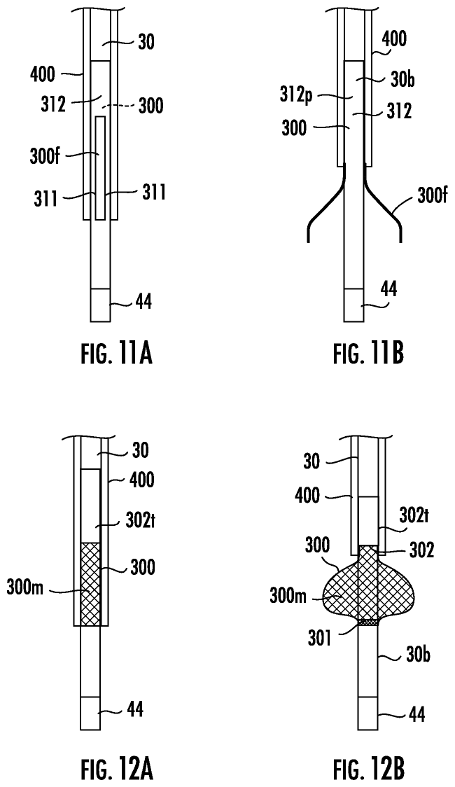

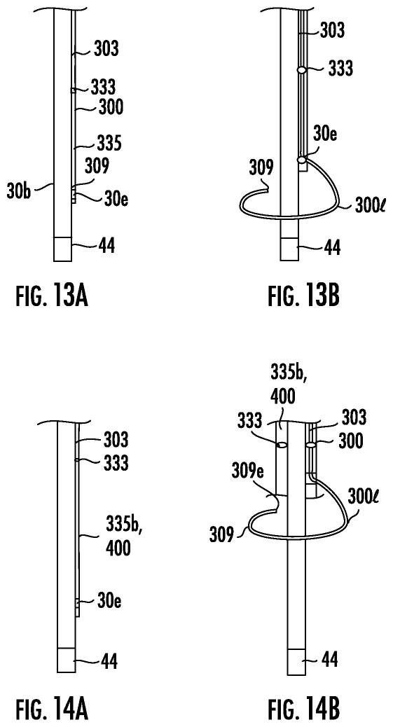

FIGS. 1C and 2C are respective partial section views of the catheter blood pump shown in FIG. 1A but with the anti-migration brace attached to an outlet cage rather than the catheter body and in an example deployed configuration according to embodiments of the present invention.

FIG. 3 is an enlarged schematic illustration of part of a catheter blood pump and example anti-migration brace having a three-dimensional mesh body shown in a deployed, enlarged configuration, sized and configured to abut a wall of an aorta of a patient adjacent the aortic valve according to embodiments of the present invention.

FIG. 4 is an enlarged schematic illustration of part of a catheter blood pump and example anti-migration brace having a three-dimensional lasso body shown in a deployed, enlarged configuration, sized and configured to abut a wall of an aorta of a patient adjacent the aortic valve according to embodiments of the present invention.

FIG. 5A is an enlarged schematic illustration of part of a catheter blood pump and example anti-migration brace having a tube attached to a catheter, the tube having deployable fingers, sized and configured to abut a wall of an aorta of a patient adjacent the aortic valve according to embodiments of the present invention.

FIG. 5B is a schematic illustration the anti-migration brace shown in FIG. 5A, shown in a non-deployed configuration separate from the catheter, with circumferentially spaced apart and longitudinally extending slits formed in the tube and defining outwardly deployable fingers according to embodiments of the present invention.

FIG. 6 is an enlarged schematic illustration of part of a catheter blood pump and example anti-migration brace having a three-dimensional spiral body shown in a deployed, enlarged configuration, sized and configured to abut a wall of an aorta of a patient adjacent the aortic valve according to embodiments of the present invention.

FIG. 7A is an enlarged schematic illustration of part of a catheter blood pump and example anti-migration brace shown in a deployed configuration, sized and configured to abut a wall of an aorta of a patient adjacent the aortic valve according to embodiments of the present invention.

FIG. 7B is a lateral section schematic view of a “double-barrel” configuration device with the brace lumen provided by a brace lumen tube attached to a multi-lumen catheter body providing the in-flow and out-flow lubrication flow path according to embodiments of the present invention.

FIG. 8 is a greatly enlarged section view of a portion of a catheter blood pump showing an outer sheath and collapsible/expandable anti-migration brace attached to an outer surface of a catheter body according to embodiments of the present invention.

FIGS. 9A and 9B are lateral section views of example multi-lumen catheter bodies, each having a brace lumen for holding the anti-migration brace according to additional embodiments of the present invention.

FIG. 10 is a greatly enlarged section view of a multi-lumen catheter body enclosing a drive/torque cable and having at least one lumen holding at least one anti-migration brace body according to embodiments of the present invention.

FIG. 11A is a schematic illustration of an anti-migration brace similar to that shown in FIG. 5A but in a non-deployed configuration according to embodiments of the present invention.

FIG. 11B is a schematic illustration of the anti-migration brace shown in FIG. 11A, but in a deployed configuration according to embodiments of the present invention.

FIG. 12A is a schematic illustration of an anti-migration brace similar to that shown in FIG. 3 but in a non-deployed configuration according to embodiments of the present invention.

FIG. 12B is a schematic illustration of the anti-migration brace shown in FIG. 12A, but in a deployed configuration according to embodiments of the present invention

FIG. 13A is a schematic illustration of an anti-migration brace similar to that shown in FIG. 4 but in a non-deployed configuration according to embodiments of the present invention.

FIG. 13B is a schematic illustration of the anti-migration brace shown in FIG. 13A, but in a deployed configuration according to embodiments of the present invention

FIG. 14A is a schematic illustration of an anti-migration brace similar to that shown in FIG. 4 but in a non-deployed configuration according to embodiments of the present invention.

FIG. 14B is a schematic illustration of the anti-migration brace shown in FIG. 14A, but in a deployed configuration according to embodiments of the present invention

### DETAILED DESCRIPTION

The present invention will now be described more fully hereinafter with reference to the accompanying drawings, in which embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like elements throughout. The abbreviation “FIG.” may be used interchangeably with “Fig.” and the word “Figure” in the specification and figures. It will be appreciated that although discussed with respect to a certain embodiment, features or operation of one embodiment can apply to others.

In the drawings, the thickness of lines, layers, features, components and/or regions may be exaggerated for clarity and broken lines (such as those shown in circuit of flow diagrams) illustrate optional features or operations, unless specified otherwise. In addition, the sequence of operations (or steps) is not limited to the order presented in the claims unless specifically indicated otherwise.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when a feature, such as a layer, region or substrate, is referred to as being “on” another feature or element, it can be directly on the other element or intervening elements may also be present. In contrast, when an element is referred to as being “directly on” another feature or element, there are no intervening elements present. It will also be understood that, when a feature or element is referred to as being “connected” or “coupled” to another feature or element, it can be directly connected to the other element or intervening elements may be present. In contrast, when a feature or element is referred to as being “directly connected” or “directly coupled” to another element, there are no intervening elements present. Although described or shown with respect to one embodiment, the features so described or shown can apply to other embodiments. The term “about” means that the noted number can vary by +/−20%.

Referring to FIGS. 1A and 2A an example blood pump 10 is shown. The blood pump 10 comprises a motor 14, a multi-lumen shaft 30 (also described as a multi-lumen catheter body) that encloses a torque or drive cable 25, an inlet cage 33 (blood intake), a (snorkel) tube 35 extending between the inlet cage 33 and an impeller 40, and an outlet cage 44 (pumped blood outlet). A snorkel 31 can be attached to the snorkel tube 35 and be positioned at a distal end 10d of the blood pump 10. The snorkel/snorkel tube may be provided in a number of configurations.

The blood pump 10 also comprises an anti-migration brace 300. FIGS. 1A and 2A show the anti-migration brace 300 in a first configuration prior to deployment. FIGS. 1B and 2B shown the anti-migration brace 300 in a second configuration after deployment. The anti-migration brace 300 has a three-dimensional body after deployment. The first configuration can be a collapsed configuration and the second configuration can be an expanded configuration that is laterally enlarged relative to the first configuration.

The first collapsed, non-deployed configuration can have a 6F-18F profile, and can be 6F, 7F, 8F, 9F, 10F, 11F, 12F, in some embodiments.

The anti-migration brace 300 can have an atraumatic configuration devoid of tissue hooks on outer ends. The anti-migration brace 300 can have closed free ends that can be rounded to define atraumatic tissue contact surfaces whereby the anti-migration brace 300 expands to abut target tissue but does not attach/hook into tissue thereat. The atraumatic configuration may reduce inflammation that might otherwise be caused by other connection configurations.

The anti-migration brace 300 can be held inside an outer sheath 400. One or both of the outer sheath 400 and the catheter body 30b can be axially slidable relative to each other so that the anti-migration brace 300 is exposed and releases the brace 300 to allow it to expand and abut target anatomy such as a wall(s) of the aorta which may be at a location that is adjacent the aortic valve. Positioning the anti-migration brace 300 in the aorta, against the aortic wall Aw, rather in the left ventricle (LV) may provide better positional stability as the LV has increased movement relative to the aorta.

In some embodiments, the anti-migration brace 300 can be attached to the catheter body 30b. When the outer sheath 400 is proximal to the brace 300, the anti-migration brace 300 expands to the enlarged second configuration.

The anti-migration brace 300 can be configured to provide a friction-fit or “clamping force” Fc (FIG. 4) against the aortic wall Aw.

FIGS. 1C and 2C illustrate the anti-migration brace 300 attached to the impeller housing 44h that can define or be attached to the outlet cage 44, rather than the catheter body 30b, and in an example deployed configuration according to embodiments of the present invention. The anti-migration brace 300 can be distal to the blood flow outlet windows 44w or proximal to the blood flow outlet windows 44w. That is, the anti-migration brace 300 can be attached to an impeller housing 44h distal to the outlet cage 44 or proximal to the outlet cage 44.

FIG. 3 shows the expandable anti-migration brace 300 can have a three-dimensional expandable mesh body 300m. The distal end portion 301 and the proximal end portion 302 of the mesh body 300m can define necks that can be affixed to the catheter 30. The medial segment of the mesh body 300m between the proximal and distal ends 301, 302, can provide the laterally expandable mesh structure. The mesh structure of the mesh body 300m can be configured with relatively large lattice network apertures so as not to promote clotting and allowing free blood flow. Anti-clotting coating and/or materials can be used to form the mesh structure of the mesh body 300m.

FIGS. 12A and 12B illustrate respective example undeployed and deployed configurations of the mesh body 300m with a portion of the catheter body 30b and outer sheath 400. The proximal end portion 302 can be attached to a tube 302t and the tube 302t can be attached to the catheter body 30b. The sheath 400 can be coaxial to the catheter body 30b.

FIG. 4 shows the expandable anti-migration brace 300 can have a three-dimensional expandable lasso body 300l. The lasso body 300l can have a single loop 309 with a free end 309e. The loop 309 can extend circumferentially 270-360 degrees or more with a free end 309e and the loop projects laterally outward to contact target anatomical structure such as the aortic wall Aw.

FIGS. 13A and 13B illustrate respective example undeployed and deployed configurations of the lasso body 300l. No outer sheath 400 is required. A brace lumen 335 in the multi-lumen catheter body 30b can be used to hold the lasso body in the non-deployed position (FIGS. 7, 13A) and the brace body can slidably extend and retract in the brace lumen 335. A leading end of the lasso body 300l that forms the free end 309e of the lasso shape can be configured to exit out a side port 30e in the catheter body 30b and a segment of the lasso body 300l proximal thereto then follows the leading end to expand and at least partially surround the catheter body 30b to form the lasso body 300l outside the brace lumen 335 (FIG. 13B). A seal 333 such as an O-ring or other sealant material and/or member can be held inside the brace lumen 335 and couple to a “wire” shaft 303 of the lasso body 300b to inhibit/prevent blood intake thereat.

FIGS. 14A and 14B illustrate another embodiment of respective example undeployed and deployed configurations of the lasso body 300l. Here an outer sheath 400 or a brace tube 335b providing the brace lumen 335 can be used to enclose the lasso body 300l when in a non-deployed state. FIG. 7B shows an example brace tube 335b providing the brace lumen 335 and attached to the catheter body 30b forming a “double barrel” configuration.

FIG. 5A shows the expandable anti-migration brace 300 can have a tube 312 attached to the catheter body 30b and having longitudinally extending, circumferentially spaced apart fingers 300f. Referring to FIGS. 5A and 5B, the tube 312 can have longitudinally extending slits 311, one on each side of a respective finger 300f. The fingers 300f can be provided as 3, 4, 5, 6, 7 or 8 fingers 300f that are circumferentially spaced apart and project outward from the catheter body 30b. The fingers 300f are circumferentially spaced apart and attached to a tube segment 312t and the tube segment 312t is attached to the catheter body 30b.

FIG. 5B shows that the fingers 300f have a non-deployed configuration whereby the fingers 300f define a cylinder 300c aligned with the tube segment 312t. Thus, first and second circumferentially spaced apart and longitudinally extending slits 311 can separate a respective finger 300f.

FIGS. 11A and 11B illustrate respective example undeployed and deployed configurations of the fingers 300f. When the sheath 400 and/or the catheter body 30b is moved to expose the fingers 300f, the fingers 300f “spring” outward to contact and clamp against local tissue. The fingers 300f have free ends 300e that can be formed to bend to extend axially, in a distal direction for an atraumatic tissue contact configuration. The tube 312 can have a proximal end portion 312p that is affixed to the catheter body 30b. The tube 312 can be provided by a shape memory alloy, such as Nitinol.

In some embodiments, for introduction into the patient body and advancing the pump to the heart, some in-vivo elements can be contained in the outer sheath 400. Once the catheter blood pump 10 is properly sited, such that, for example, the distal portion of the housing is disposed in the left ventricle, and the proximal portion of the housing is in the ascending aorta, the housing crossing the aortic valve, the sheath 400 is partially withdrawn. More particularly, as the sheath 400 is drawn away from the heart, the deployable brace 300 is exposed. As the brace 300 is freed from the confines of the sheath 400, it automatically “springs” into shape and position. Optionally, its temperature may increase from exposure to blood which may facilitate it expanding, extending to abut against the wall of the aorta. This “friction fit” against the wall of the aorta fixes the catheter blood pump in place and/or limits axial movement.

In other embodiments, a wire 303 of the anti-migration brace 300 can be moved to deploy and retract the brace 300, without requiring a sheath 400 or without moving the sheath 400.

The deployable brace 300 may comprise an expandable material, such as a shape memory alloy (SMA) and/or nickel titanium, also known as “Nitinol.” In some embodiments, the deployable brace can comprise a plastic material. In some embodiments, the SMA and/or Nitinol is coated with a material that provides at least one of the following benefits: (i) is softer on the arterial wall than Nitinol, (ii) inhibits cell growth, (iii) reduce thrombus/blood clots. With respect to item (ii), the desire is to inhibit cell growth that might otherwise encapsulate the Nitinol, etc., in the arterial wall. The coating material can be a suitably selected grade of PEBAX® MED brand polyether block amide, commercially available from Arkema Inc., King of Prussia, PA.

The deployable brace 300 can be recaptured in the catheter 30 and/or internal to the sheath 400, or brace lumen tube 335b, to withdraw the catheter blood pump from a patient.

FIG. 6 shows the expandable anti-migration brace 300 can have a spiral body 300s, with a plurality of circumferentially extending loops 309, at least some of the loops 309 radially increase in size axially, from a proximal end portion to distal end portion.

FIG. 7A illustrates that the blood pump 10 can have a torque cable 25 with a length sufficient to position the motor 14 to be extracorporeal while the expandable brace 300 resides in the aorta. The blood pump 10 can also include a controller with a user interface 177. A wire shaft 303 of the anti-migration brace 300 can be coupled to or accessible by the UI 177.

FIG. 8 shows the expandable anti-migration brace 300 can have a proximal end portion 302 that is attached to the catheter body 30b, typically the outer wall 30w of the catheter body 30b. The outer sheath 400 can be slidably held over the catheter body 30b and may be flexible to be conformal to the shape of the catheter body 30b. The outer sheath 400 can have a length sufficient to cover some in vivo components of the blood pump 10, typically terminating proximal to the outlet cage 44.

FIG. 9A shows a multi-lumen catheter 30 with at least one brace lumen 335 configured to slidably hold the anti-migration brace therein. FIG. 9A shows the at least one brace lumen 335 formed in a catheter body 30b and extending longitudinally to a discharge area closer to the impeller 40. FIG. 9B shows the at least one brace lumen 335 having an annular shape and surrounding the torque cable 25.

The multi-lumen catheter 30 (which can also referred to interchangeably as a “multi-lumen shaft”) is shown with a plurality of internal lumens 131, 133, 135, 335. In this view, the (blood) outflow cage 44 is also shown, but it is not part of the body 30b of the multi-lumen shaft 30. The body 30b of the multi-lumen shaft 30 can be provided as an extruded body 30b with multiple longitudinally extending lumens 131, 133, 335 and the one or more ports 130 extending through the outer wall 30w to the in-flow lumen(s) 133. The body 30b can be an extruded body of polyamide or polyimide.

A separate tube 131t, such as a PEBAX tube, can be used to provide the lumen 131 that encases the torque cable 25 and provide at least a portion of the (fluid purge) outflow Fo path. Alternatively, the lumen 131 can be directly formed in the body 30b of the multi-lumen shaft 30. The at least one in-flow lumen 133 can be provided as a pair of diametrically opposed lumens as shown. The at least one in-flow lumen(s) 133 can be provided as a plurality of separate tubes or passages directly formed in the multi-lumen shaft body 30b. The at least one in-flow lumen 133 can be provided as polymer tubes (optionally polyimide tubes) 133t.

FIG. 9B illustrates another example extruded body 30b′ with an in-flow lumen 133 that is provided as a ring surrounding the out-flow lumen 131 and the brace lumen 335 surrounding the in-flow lumen 133, arranged to provide concentric or coaxial lumen configurations.

FIG. 10 shows the at least one brace lumen 335 with the expandable brace 300 held outside an inflow path 133. At least part, typically at least 50% of an overall length of the brace shaft 303, can remain in the respective brace lumen 335 when a distal end portion of the brace 300 is outside the lumen 335 and in an expanded configuration. The shaft 303 can be axially retracted or extended in the respective lumen 335 to contain and deploy, respectively, the brace 300.

Referring to FIGS. 1A-1C and 2A-2C, the blood pump 10 can have a manifold 110 that is coupled to the motor 14. The manifold 110 has a manifold chamber 110c. The manifold 110 can sealably enclose a sub-length of the shaft 30, typically at least a segment of the proximal end portion 30p of the multi-lumen shaft 30 and can define at least a portion of a (purge) fluid in-flow path into at least one aperture/port 130 in an outer wall 30w of the multi-lumen shaft 30, then into at least one in-flow lumen(s) 133 provided by the multi-lumen shaft 30. The term “in-flow” can be used interchangeably with the term “inflow” herein. The term “out-flow” can be used interchangeably with the term “outflow” herein.

Referring to FIGS. 1A, 1B, 8 and 10, the blood pump 10 can also have a bearing housing 50 adjacent the impeller 40 with a bearing housing adapter 52 that couples an outer wall 30w of the multi-lumen shaft 30 to the bearing housing 50. The bearing housing 50 can comprise a lateral, cross-flow passage 55 that is in fluid communication with an a radially extending passage 602 of a bearing/bushing 600 and a longitudinal channel 605 thereof, that provides part of the out-flow path Fo.

The multi-lumen catheter/shaft 30 has a proximal end portion 30p that is adjacent the motor 14 and an opposing distal end portion 30d that terminates adjacent the impeller 40. The torque cable 25 also has a proximal end portion 25p that is adjacent the motor 14 and an opposing distal end portion 25d that terminates adjacent the impeller 40. The torque cable 25 can also be interchangeably referred to as a “drive cable”. The torque cable 25 can be directly or indirectly attached to the impeller 40 at the distal end portion 25d of the torque (drive) cable 25 and to the motor 14 at the proximal end portion 25p of the torque (drive) cable 25.

The motor 14 can be held in a housing 16. The housing 16 can be provided as a cooperating pair of handle shells 16s.

An intrabody portion of the blood pump 10 (distal to the housing 16) is configured to be inserted into the aorta from a remote entry point, such as an incision below the groin that provides access into a femoral artery. The intrabody portion of the blood pump 10 (snorkel 31 leading the way), then passes through the descending aorta until it reaches the ascending aorta, near the heart. The multi-lumen shaft 30 encloses the torque cable 25 and can have a length sufficient to position the motor 14 to be extracorporeal. The proximal end portion 30p of the multi-lumen shaft 30 can reside outside the body, typically near the patient's groin, at an end portion opposing the impeller 40 and snorkel 31.

The blood pump 10 can comprise first and second support wires 119, 219 that are longitudinally spaced apart and reside inside the torque cable 25. Referring to FIG. 1A, the first support wire 119 can have a distal end 119e that terminates a range of 1-3 inches from the manifold 110 and extends at least partially through the motor shaft. The second support wire 219 can have a proximal end 219e that terminates a range of 1-3 inches from the proximal end of the impeller shaft 140. The first support wire 119 can support the torque cable 25 at a high torque area (at the motor 14) so that the torque cable 25 does not collapse under load. The first support wire 119 can also act as a strain relief when it exits a distal end of the manifold 110. The second support wire 219 can allow the impeller shaft 140 and torque cable 25 to be crimped together by using the proximal bushing 1400 without collapsing the (hollow) torque cable 25. The second support wire 219 can also act as a strain relief.

In some embodiments, the first and second support wires 119, 219 can be provided as a single support wire instead of separate support wires and the single support wire may extend substantially an entire length of the torque cable 25 or reside only at a proximal end portion or only at a distal end portion of the torque cable 25.

Referring to FIGS. 8 and 10, the at least one in-flow lumen 133 can extend a distance “D” distally of a distal end 131d of the torque cable lumen 131. Stated differently, the torque cable lumen 131 can terminate proximally a distance from a distal end of the inflow lumen 133. The distance “D” can be in a range of 10-30% of a length of the impeller shaft 140. The distance D can be in a range of 0.1 mm to 0.3 mm, in some embodiments. The impeller shaft 140 can define a portion of the outflow path Fo extending between the cross-over Fc purge fluid flow path to the distal end 131d of the torque cable lumen 131. The impeller shaft 140 and configurations of the inflow and outflow lumens 133, 131, respectively can provide structural rigidity/durability to the multi-lumen shaft 30.

Referring to FIGS. 8 and 10, enlarged section views of a portion of the blood pump 10 that is adjacent the impeller 40. As shown, the impeller 40 is attached to an impeller shaft 140 that extends out of a distal end portion 30d of the multi-lumen shaft 30. A proximal bushing 1400 and a distal bushing 500 can sandwich an intermediate bushing 600 (sometimes referred to as a “bearing” but functionally, this component does not have “bearings” or movable components that rotate so that “bushing” may be a more accurate term). Also, for ease of discussion in light of the proximal and distal bushings, the term “bearing/bushing”, “bearing (bushing)”, or “bushing/bearing” and the like 600 can be used herein interchangeably to refer to the bushing 600 component.

The bearing/bushing 600 can be formed of a polymer such as PEEK. In some embodiments, the bearing/bushing 600 can be formed of 3016 PEEK, with carbon fibers in a range of 15-25% (by volume carbon fiber and PTFE in a range of 15-25% (by volume) thereby providing a wear resistant and lubricious body.

FIG. 7A illustrates an example operational configuration with the blood intake cage 33 and snorkel 31 in a left ventricle (LV) of a heart of a patient, the anti-migration brace 300, the impeller 40 and blood outlet cage 44 positioned in the aorta, proximate the coronary arteries, above the aortic valve to discharge pumped blood into the aorta, while the motor 14 and motor housing 16 are outside the patient. However, it is noted that the motor may be directly coupled to the impeller and reside in the body.

Generally stated, when the proximal end portion of the torque cable 25 is mechanically rotated by a motor shaft of the motor 14, typically located outside the patient's body, it conveys the rotational force through the length of the multi-lumen shaft 30, causing the impeller 40 to spin at high speed near the heart.

The blood pump 10 can be particularly suitable in providing ventricular assist during surgery or providing temporary bridging support to help a patient survive a crisis.

The motor 14 is arranged to drive the torque cable 25 in the multi-lumen shaft 30 which in turn drives the impeller 40/pump unit. The motor 14, being operated at an extracorporeal site, can have any desired size. The multi-lumen shaft 30 provides continuous lubrication by a biocompatible (purge) liquid. A part of this liquid can exit through a bearing housing/impeller shaft interface and thus enter the blood stream. The remaining part can be directed to flow through an out-flow path and be collected extracorporeally after passing through a lumen 131 provided in the multi-lumen shaft 30 that holds the drive cable 25.

Referring to FIGS. 8 and 10, a portion of the proximal bushing 1400 and the proximal end of the impeller shaft 140 can reside inside the out-flow lumen 131 and/or inside the out-flow tube 131t, in-line with the torque cable 25.

The bearing housing 50 can hold at least a portion of the bearing/bushing 600. The bearing housing 50 can also have first and second longitudinally extending channels 53 on opposing sides of the center channel 54. The first and second channels 53 are in fluid communication with and/or hold a segment of a respective in-flow tube 133t. The bearing housing 50 can have lateral passages 55 that are in fluid communication with the ports 602 of the bearing/bushing 600, and the longitudinally extending channel 605. The lateral passages 55 can fluidly connect the in-flow path F₁ provided by the in-flow lumens 133 with the outflow path Fo provided by the torque cable lumen 131, using the bearing/bushing 600. Seal material (such as adhesive) 155 can be used to seal outer ends of the passages 55. The lateral passages 55 can be aligned with the ports 602 of the bearing/bushing 600.

The lateral passages 55 can have an outer diameter in a range of 0.010 inches to about 0.030 inches, such as about 0.020 inches. The first and second channels 53 can have an outer diameter that is greater than the lateral passages 55, typically in a range of about 0.020 inches to about 0.030 inches, such as about 0.025 inches, in some embodiments.

The deployable brace 300 can be disposed on the exterior of the catheter 30, typically at one or more centimeters away from a proximal end of the impeller cage or housing 44. This position can ensure that the impeller housing 44 remains centered between the valve flaps and that the brace 300 will not interrupt blood flow into the aorta.

Referring to FIG. 7, the anti-migration brace 300 can have a maximal laterally outward projection Pm at a location D that can be in a range of 5 mm to 4 cm proximal to a proximal end 40p of the impeller 40 and/or proximal end 44p of the outlet cage 44.

It is to be understood that the disclosure describes a few embodiments and that many variations of the invention can easily be devised by those skilled in the art after reading this disclosure and that the scope of the present invention is to be determined by the following claims.

The blood pump 10 can be sized and configured for trans-valvular use, such as for left and/or right ventricular assist procedures. By way of example only, such ventricular assist procedures may be employed in cardiac operations including, but not limited to, coronary bypass graft (CABG), cardiopulmonary bypass (CPB), open chest and closed chest (minimally invasive) surgery, bridge-to-transplant and/or failure-to-wean-from-bypass situations. It is to be readily understood, however, that the intravascular blood pump assembly and methods of the present invention are not to be limited to such applications. Moreover, while illustrated and described largely with reference to left-heart assist applications, it is to be readily understood that the principles of the present invention apply equally with regard to right-heart assist application, which are contemplated as within the scope of the present invention. These and other variations and additional features will be described throughout.

The blood pump 10 can be configured to pump blood through the outlet cage 44 at a rate in a range of 2-7 liters/minute over at least 6 days of continuous intravascular use while continuously providing biocompatible fluid to the in-flow path FI via at least one in-flow lumen 133, then to the out-flow path Fo.

The blood pump 10 may be configured to provide axial or mixed-flow. As used herein, the term “axial flow” is deemed to include flow characteristics which include both an axial and slight radial component.

The multi-lumen shaft 30 and the impeller 40 may be dimensioned to any suitable diameter for intravascular applications. For example, the range of sizes may include, but is not necessarily limited to, 9 French to 30 French, although the range is typically in a range of 12 French to 24 French, and more typically in a range of 12 French to 18 French. Cardiologists can thus insert the small CBP device minimally invasively. In some preferred embodiments, the diameter of can be about 12 French (4.0 mm) or less providing a low-profile device that can minimize bleeding.

The blood pump 10 can be configured to provide right and/or left heart support whereby blood is deliberately re-routed through and past the right and/or left ventricle in an effort to reduce the volume of blood to be pumped by the particular ventricle. While “unloading” the ventricles in this fashion is preferred in certain instances, it is to be readily understood that the pump and cannula arrangements described herein may also be employed to “preload” the ventricles. Ventricular preloading may be accomplished by positioning the outflow cage from the pump into a given ventricle such that the pump may be employed to fill or preload the ventricle with blood. This may be particularly useful with the right ventricle. On occasion, the right ventricle is not supplied with sufficient levels of blood from the right atrium such that, upon contraction, the right ventricle delivers an insufficient quantity of blood to the pulmonary artery. This may result when the right ventricle and/or right atrium are in a stressed or distorted condition during surgery. Preloading overcomes this problem by actively supplying blood into the right ventricle, thereby facilitating the delivery of blood into the pulmonary artery. The same technique can be used to preload the left ventricle and thus facilitate the delivery of blood from the left ventricle into the aorta.

In the drawings and specification, there have been disclosed embodiments of the invention and, although specific terms are employed, they are used in a generic and descriptive sense only and not for purposes of limitation, the scope of the invention being set forth in the following claims.

Thus, the foregoing is illustrative of the present invention and is not to be construed as limiting thereof. More particularly, the workflow steps may be carried out in a different manner, in a different order and/or with other workflow steps or may omit some or replace some workflow steps with other steps. Although a few exemplary embodiments of this invention have been described, those skilled in the art will readily appreciate that many modifications are possible in the exemplary embodiments without materially departing from the novel teachings and advantages of this invention.

What is claimed:

1. A catheter blood pump comprising:
an impeller;
an outlet cage at least partially surrounding the impeller;
a drive cable operatively coupled to a motor at a proximal end thereof, and operatively coupled to the impeller at a distal end thereof;
a catheter, wherein the drive cable is disposed within the catheter; and
an anti-migration brace coupled to a component of the catheter blood pump, wherein the anti-migration brace is configured to have a first configuration during a tortuous insertion of the catheter into a patient and a deployable second configuration that is larger than the first configuration when in position in target anatomy of the patient whereby the anti-migration brace expands laterally outwardly in size from the first configuration to the second configuration.

2. The catheter blood pump of claim 1, wherein, in the second configuration, the anti-migration brace increases in size in a longitudinal direction from a first position to a second more distal position closer to the impeller.

3. The catheter blood pump of claim 1, wherein the anti-migration brace is disposed on an exterior of the catheter, proximal to the outlet cage.

4. The catheter blood pump of claim 1, further comprising a sheath enclosing the anti-migration brace when the anti-migration brace is in the first configuration, wherein the sheath is configured to force the anti-migration brace against the component of the catheter blood pump and provide a profile of 6F-18F in the first configuration.

5. The catheter blood pump of claim 1, wherein the catheter has a multi-lumen catheter body and comprises at least one longitudinally extending brace lumen, and wherein a distal end portion of the anti-migration brace is configured to be extendable out of the at least one longitudinally extending brace lumen and retractable back into the at least one longitudinally extending brace lumen.

6. The catheter blood pump of claim 1, wherein the anti-migration brace is attached to an outer surface of the catheter at a location that is a distance proximal to the impeller and that is sized and configured to reside about an aortic wall of an ascending aorta of the patient in the second configuration.

7. The catheter blood pump of claim 6, wherein the anti-migration brace has a maximal projection at a location that is in a range of 5 mm-4 cm proximal to a proximal end of the impeller.

8. The catheter blood pump of claim 1, wherein the anti-migration brace is attached to an impeller housing distal to the outlet cage.

9. The catheter blood pump of claim 1, wherein the anti-migration brace is attached to an impeller housing proximal to the outlet cage.

10. The catheter blood pump of claim 1, wherein the anti-migration brace comprises an expandable mesh body with proximal and distal end portions configured to attach to the component of the catheter blood pump and a medial segment therebetween configured to expand into a three-dimensional shape in the second configuration.

11. The catheter blood pump of claim 1, wherein the anti-migration brace comprises an expandable lasso.

12. The catheter blood pump of claim 11, wherein the expandable lasso has a single loop.

13. The catheter blood pump of claim 11, wherein the expandable lasso has an open loop configuration.

14. The catheter blood pump of claim 1, wherein the anti-migration brace comprises a plurality of fingers that project radially outward from the catheter when deployed.

15. The catheter blood pump of claim 14, wherein the fingers are circumferentially spaced apart and attached to a tube segment, and wherein the tube segment is attached to the catheter.

16. The catheter blood pump of claim 15, wherein in the first configuration, the fingers define a cylinder aligned with the tube segment.

17. The catheter blood pump of claim 16, wherein the anti-migration brace is provided by a tubular body with first and second circumferentially spaced apart and longitudinally extending slits separate a respective finger.

18. The catheter blood pump of claim 15, wherein the plurality of fingers are provided in a number of 3 to 8 fingers.

19. The catheter blood pump of claim 1, wherein the anti-migration brace comprises a spiral member with a series of circumferential segments that increase in radius from a proximal end to a more distal end and that have radial extents that are greater than an outer diameter of a catheter body.

20. The catheter blood pump of claim 1, wherein the anti-migration brace has an atraumatic configuration with tissue contact surfaces that are devoid of tissue hooks.

21. The catheter blood pump of claim 4, wherein on introduction of the catheter blood pump into the patient, the sheath contains the at least part of the catheter and the anti-migration brace, and wherein the anti-migration brace is physically adapted to automatically expand when the sheath is partially slidably withdrawn relative to the catheter or the catheter is partially slidably extended relative to the sheath.
